(19) United States
(12) Patent Application Publication
(10) Pub. No.: US 2008/0031955 A1
(43) Pub. Date: Feb. 7, 2008

(54) Product Comprising a Beta-Lactam Antibiotic

(76) Inventors: Marinus Petrus Wilhelmus Maria Rijkers, Elsloo (NL); Jordi Savall Sayago, Llica d'Amunt (ES); Miguel Sanz Pedrosa, Barcelona (ES)

Correspondence Address: NIXON & VANDERHYE, PC, 901 NORTH GLEBE ROAD, 11TH FLOOR, ARLINGTON, VA, 22203, US

(21) Appl. No.: 11/630,269

(22) PCT Filed: Jun. 28, 2005
(86) PCT No.: PCT/EP05/53035

§ 371 Date: Dec. 20, 2006

(30) Foreign Application Priority Data
Jun. 30, 2004 (EP) 04077518.1

(51) Int. Cl. A61K 31/545; A61K 31/43; A61K 9/14; A61K 9/16; A61P 31/00

(52) U.S. Cl. 424/489; 514/198; 514/200

(57) ABSTRACT

The present invention relates to a product comprising a β-lactam antibiotic, for instance a cephalosporin, said product containing less than 200 mg of acetone per kg of β-lactam antibiotic. The invention also relates to a process for drying a β-lactam antibiotic, for instance a cephalosporin, said process comprising drying said β-lactam antibiotic under decreased pressure.

The present invention relates to a product comprising a β-lactam antibiotic. The invention also relates to a process for drying a β-lactam antibiotic.

The preparation of a product comprising a β-lactam antibiotic typically involves a drying step. In industry fluid bed dryers are known for this purpose. A β-lactam antibiotic can be washed prior to said drying. For instance, a process is known wherein the β-lactam antibiotic, for instance a cephalosporin, is washed with acetone prior to drying with a fluid bed drier. Washing with acetone is advantageous since it is found to result in a dried product having improved quality, such as for instance expressed in absorbance, colour, and the like. However, the dried product obtained in this manner can contain residual amounts of acetone, for instance 500 mg per kg of β-lactam antibiotic. This is disadvantageous as acetone is an alien substance.

The invention provides a drying process wherein dried product can be obtained having significant smaller amounts of acetone (or organic solvents in general) or even no acetone or even no organic solvent, and still having a good quality. Accordingly, the invention provides a product comprising a β-lactam antibiotic, said product containing less than 200 mg of acetone per kg of β-lactam antibiotic. Preferably, the product according to the invention contains less than 100 mg, preferably less than 80 mg, preferably less than 50 mg, preferably less than 20 mg, preferably less than 10 mg of acetone per kg of β-lactam antibiotic. Some residual acetone may remain. In an embodiment, the product according to the invention contains above 1 mg of acetone per kg of β-lactam antibiotic. In another embodiment, the product according to the invention contains no acetone.

The process according to the invention allows obtaining dried β-lactam antibiotic in solid form having good quality.

In another aspect, the invention provides a product comprising a β-lactam antibiotic, said product containing less than 200 mg, preferably less than 100 mg, preferably less than 80 mg, more preferably less than 50 mg, more preferably less than 20 mg, more preferably less than 10 mg of an organic solvent selected from a ketone or an alcohol, per kg of β-lactam antibiotic. Some residual organic solvent selected from the group consisting of ketone and alcohol may remain. In an embodiment, the product according to the invention contains above 1 mg of organic solvent selected from the group consisting of ketone and alcohol per kg of β-lactam antibiotic. In another embodiment, the product according to the invention contains no organic solvent selected from the group consisting of ketone and alcohol per kg of β-lactam antibiotic.

In another aspect, the invention also provides a product comprising a β-lactam antibiotic, said product containing less than 200 mg, preferably less than 100 mg, preferably less than 80 mg, more preferably less than 50 mg, more preferably less than 20 mg, more preferably less than 10 mg of organic solvent per kg of β-lactam antibiotic. Some residual organic solvent may remain. In an embodiment, the product according to the invention contains above 1 mg of organic solvent per kg of β-lactam antibiotic. In another embodiment, the product according to the invention contains no organic solvent.

The acetone content or concentration of organic solvent may be determined in any suitable manner, e.g. by gas chromatography.

The product according to the invention may be any β-lactam in solid form having the decreased concentration of acetone and/or decreased concentration of organic solvent according to the invention.

The product according to the invention may be a crystalline powder of said β-lactam antibiotic, hereinafter also referred to as crystalline powder according to the invention.

The product according to the invention may be in the form of granules comprising the β-lactam antibiotic, hereinafter also referred to as granules according to the invention. Granules according to the invention may comprise auxiliaries or may be free of auxiliaries. Granules according to the invention may comprise compressed β-lactam antibiotic, for instance β-lactam antibiotic compressed by roller compacting. Compressed β-lactam antibiotic may be compressed crystalline powder of the β-lactam antibiotic, for instance crystalline powder according to the invention, or a compressed mixture, said mixture comprising crystalline powder of the β-lactam antibiotic, for instance crystalline powder according to the invention, and auxiliaries. The granules according to the invention may for instance have a bulk density of between 0.4 and 1.0 g/ml, for instance between 0.45 and 0.8 g/ml.

The product according to the invention may also be a (pharmaceutical) composition comprising the β-lactam antibiotic. The product according to the invention may be a mixture comprising (i) crystalline powder according to the invention and/or granules according to the invention and (ii) auxiliaries. The composition may be a capsule or sachet containing a β-lactam antibiotic, for instance granules according to the invention, and, optionally, auxiliaries. The composition may also be a tablet comprising a tabletted mixture comprising granules according to the invention and optionally auxiliaries.

The invention also provides a process comprising mixing crystalline powder according to the invention with auxiliaries. The invention also provides a mixture comprising crystalline powder according to the invention and auxiliaries.

As auxiliaries may for instance be used fillers, dry binders, disintegrants, wetting agents, wet binders, lubricants, flow agents and the like. Examples of auxiliaries are lactose, starches, bentonite, calcium carbonate, mannitol, microcrystalline cellulose, polysorbate, sodium lauryl sulphate, carboxymethylcellulose Na, sodium alginate, magnesium stearate, silicon dioxid, talc.

The invention also provides compressed products, for instance granules or tablets, comprising compressed crystalline powder according to the invention or comprising a compressed mixture comprising crystalline powder according to the invention and auxiliaries. The invention also provides capsules are sachets containing said granules.

The invention also contemplates feeding crystalline powder according to the invention or a mixture comprising crystalline powder according to the invention and auxiliaries to a roller compactor to form compacts; and milling the compacts to produce granules. The invention also provides granules obtainable by said process. The granules may advantageously be sieved to obtain a desired particle size distribution. The granules may for instance have a bulk density of between 0.4 and 1.0 g/ml, for instance between 0.45 and 0.8 g/ml.

The process provided by the invention allows obtaining a dried product having a good quality.

In a preferred embodiment, the product according to the invention has a C₄₀₀≦0.20, wherein C₄₀₀ is the color measured at a wavelength of 400 nm (Ph.Eur.). Preferably, the product has a C₄₀₀≦0.15, preferably a C₄₀₀≦0.10.

In another embodiment, the product according to the invention has an A₃₃₀≦0.05, preferably an A₃₃₀≦0.04, preferably an A₃₃₀≦0.03, preferably an A₃₃₀≦0.02, A₃₃₀≦0.01, wherein A₃₃₀ is the absorbance measured at a wavelength of 330 nm (Ph.Eur).

In another embodiment, the product according to the invention, wherein for instance the β-lactam antibiotic is cephalexin, has an A₂₆₂ of between 220 and 245, wherein A₂₆₂ is the absorbance measured at a wavelength of 262 nm. (Ph. Eur.).

In another embodiment, the product according to the invention, wherein for instance the β-lactam antibiotic is cephradin, has an A₂₆₂ of between 220 and 245, wherein A₂₆₂ is the absorbance measured at a wavelength of 262 nm. (Ph. Eur.).

In another embodiment, the product according to the invention, wherein for instance the β-lactam antibiotic is cephradin, has an A₄₅₀≦0.03, preferably A₄₅₀≦0.02, preferably A₄₅₀≦0.01, wherein A₄₅₀ is the absorbance measured at a wavelength of 450 nm. (Ph. Eur.).

In another embodiment, the product according to the invention, wherein for instance the β-lactam antibiotic is cefadroxil, has a A₂₆₄ between 225 and 250, wherein A₂₆₄ is the absorbance measured at a wavelength of 264 nm (Ph. Eur).

In an embodiment, the product according to the invention has a water content (Karl Fisher) of less than 15%, for instance less than 8%, for instance above 4.0%.

In an embodiment, the β-lactam antibiotic is cephalexin, and the product according to the invention has a water content (Karl Fisher) of between 4.0 and 8.0%.

In an embodiment, the β-lactam antibiotic is cefadroxil, and the product according to the invention has a water content (Karl Fisher) of between 4.2 and 6.0%.

The invention also provides a method for preparing the product according to the invention.

The invention provides a process for drying a β-lactam antibiotic, said process comprising drying said β-lactam antibiotic under decreased pressure. The invention also provides a process for drying a β-lactam antibiotic, said process comprising stirring said β-lactam antibiotic during said drying, preferably using mechanical means.

Most preferably, the invention provides a process for drying a β-lactam antibiotic, said process comprising drying said β-lactam antibiotic under decreased pressure and stirring said β-lactam antibiotic during said drying. This combination according to the invention is found to result in strongly decreased concentrations of acetone and/or of organic solvent in the dried product.

Drying the β-lactam antibiotic under reduced pressure may be effected at any pressure below 1 bar, for instance between 1 and 500 mbar, preferably between 5 and 200 mbar, more preferably between 10 and 100 mbar. Drying may be effected at any suitable temperature, for instance between 20 and 100° C., for instance between 40 and 80° C.

Stirring said β-lactam antibiotic during said drying may be effected in any suitable manner, preferably using mechanical means. As mechanical means an agitator may be used that may be present in the drying chamber. The agitator may be rotated around an axis in the drying chamber. Said agitator may comprise blades, for instance connected to the axis.

Drying may suitably be effected in a contact dryer. Drying may be effected in a dryer having any suitable capacity, for instance a capacity of between 0.1 m³ to 100 m³, for instance between 0.5 m³ to 50 m³.

Surprisingly, it was found that drying a β-lactam antibiotic in a process comprising mechanically impacting the β-lactam antibiotic during said drying, for instance stirring the β-lactam antibiotic during said drying can result in crystalline powder of said β-lactam antibiotic having an increased bulk density. This was particularly found when the β-lactam antibiotic comprises crystals having an aspect ratio (longest dimension of the crystal divided by the smallest dimension of the crystal) above 2, for instance above 3, for instance cephalexin monohydrate crystals. Without wishing to be bound by any scientific theory it is believed that mechanically impacting the crystals result in breaking of the crystals, this resulting in increased values of the bulk density.

Accordingly, the invention also provides a process for drying a β-lactam antibiotic comprising crystals having an aspect ratio above 2 or above 3, for instance cephalexin monohydrate, said process comprising mechanically impacting the β-lactam antibiotic during said drying. Preferred embodiments are described herein above.

Accordingly, the invention also provides crystalline cephalexin monohydrate powder having a bulk density of above 0.25 g/ml, for instance above 0.28 g/ml, preferably above 0.30 g/ml, more preferably above 0.35 g/ml. Crystalline cephalexin monohydrate powder having a bulk density above these preferred values is believed to be new and is an aspect of the invention. There is no specific upper limit for the bulk density. In practice, the bulk density may for instance be below 0.5 g/ml. As used herein, bulk density is preferably determined using USP 24, method I, (page 1913). Preferably, bulk density is determined using method Eur. Ph. 5.0, section 2.9.15.

Crystalline cephalexin monohydrate powder according to the invention may have any suitable value for d₅₀, for instance between 1 and 100 μm, for instance between 2 and 50 μm. A preferred way for determining d₅₀ is laser diffraction, for instance by using Malvern equipment. Crystalline powder having increased bulk density according to the invention may advantageously be used to prepare granules, for instance by roller compacting, for instance using one of the process described hereinabove.

In an embodiment of the invention the process comprises drying a β-lactam antibiotic containing an organic solvent, for instance acetone, an alcohol, or dichloromethane. The organic solvent may be present as a result of washing the β-lactam antibiotic with an organic solvent or with a solution containing an aqueous solvent, prior to said drying. In an embodiment, the process comprises washing the β-lactam antibiotic with an organic solvent or a solution containing an aqueous solvent, prior to said drying. Said organic solvent may be a ketone, for instance acetone, or an alcohol. When the β-lactam antibiotic is a cephalosporin, the organic solvent is preferably acetone. By applying the drying process according to the invention, it was also found to be possible to obtain good product quality when the β-lactam antibiotic is washed with water prior to said drying. This allows product to be obtained containing no acetone or a product containing no organic solvent selected from the group consisting of ketone and alcohol, or a product containing no organic solvent. Therefore, in an embodiment, the process according to the invention also comprises washing the β-lactam antibiotic with water prior to said drying.

Dichloromethane may be present as a result of a chemical process for preparing the β-lactam antibiotic.

The β-lactam antibiotic is not limited to a specific type of β-lactam antibiotic. It may for instance be a penicillin, for instance ampicillin or amoxicillin, or a cephalosporin, for instance cephalexin, cefadroxil, cephradin or cefaclor.

Cephalexin may be in any suitable form, for instance in the form of a hydrate, for instance cephalexin monohydrate.

Cefadroxil may be in any suitable form, for instance in the form of a hydrate, for instance cefadroxil monohydrate.

Cephradin may be in any suitable form, for instance in the form of a hydrate, for instance cephradin monohydrate.

Cefaclor may be in any suitable form, for instance in the form of a hydrate, for instance cefaclor monohydrate.

Amoxicillin may be in any suitable form, for instance in the form of a hydrate, for instance amoxicillin trihydrate.

Ampicillin may be in any suitable form, for instance in the form of a hydrate, for instance ampicillin trihydrate.

The β-lactam antiobiotic may be prepared in any suitable process known in the art, for instance using a chemical process or an enzymatic process.

The invention will be further elucidated by means of the following examples, without however being limited thereto.

### EXAMPLES

In all examples and comparative experiments, cephalexin was prepared and recovered using the process as described in WO-A-9623796. The cephalexin (monohydrate) crystals obtained were washed with water and subsequently with a water-acetone mixture containing 80 vol. % of acetone. The resulting wet cake contained 8 wt.% of free water and 8 wt. % of acetone.

### Comparative Experiment A

A fluid bed dryer was charged with 130 kg of cephalexin wet cake produced as described above, and containing 8 wt. % of free water and 8 wt. % of acetone. The wet cake was fluidised with air at ambient temperature during 15 to 30 minutes, after which it was fluidised with warm air (30-40° C.) during 45 to 75 minutes (air flow 1800 m³/hr), until the water content was 5.2 wt. % (Karl Fisher).

The acetone content of the dried product was analysed using a Hewlett-Packard Gas Chromatograph, model 5890. As a sample solution was used a solution obtained by dissolving 0.500 g of product in 10.0 ml of water (with the aid of some drops of NaOH 1 N). As a standard (reference) solution was used a solution obtained by dissolving 0.5 g of acetone standard in 50.0 ml of water, from which 1.0 ml was taken and diluted with 50.0 ml water.

The acetone content determined in this manner was 500 mg of acetone per kg of cephalexin.

The bulk density of the product was determined using method Eur. Ph. 5.0, section 2.9.15 (with the difference that a 100 ml cylinder was used).

The results are indicated in table 1.

### Example 1

Example 1 was repeated with the difference that drying was carried out using a Vacuum Paddle dryer type SHV-3000 supplied by Bachiller S. A., Spain. The dryer was charged with 600 kg cephalexin wet cake produced as described above, containing 8 wt. % of free water and 8 wt. % of acetone. The walls were heated at a temperature of 70° C. (product temperature 40° C.). The final pressure was 20 mbar. During drying the wet cake was stirred at a speed of 7 rpm. After 2 hours and 40 minutes of drying the product was discharged. The water content was 5.2 wt. % (Karl Fisher). The bulk density of the product was determined using method Eur. Ph. 5.0, section 2.9.15 (with the difference that a 100 ml cylinder was used). The results are indicated in table 1.

TABLE 1

| | Comparative experiment A | Example I |
|---|---|---|
| Acetone content (mg/kg) | 500 | 10 |
| C₄₀₀ (Ph. Eur.) | 0.025 | 0.026 |
| Bulk density (g/ml) | 0.27 | 0.36 |

1. Product comprising a β-lactam antibiotic, for instance a cephalosporin, said product containing less than 200 mg of acetone per kg of β-lactam antibiotic.

2. Product according to claim 1, wherein said product contains less than 100 mg, preferably less than 80 mg, more preferably less than 50 mg, more preferably less than 20 mg, more preferably less than 10 mg of acetone per kg of β-lactam antibiotic.

3. Product, for instance according to claim 1, wherein said product contains less than 200 mg, preferably less than 100 mg, preferably less than 80 mg, more preferably less than 50 mg, more preferably less than 20 mg, more preferably less than 10 mg of an organic solvent selected from the group consisting of ketone and alcohol, per kg of β-lactam antibiotic.

4. Product, for instance according to claim 1, wherein said product contains less than 200 mg, preferably less than 100 mg, preferably less than 80 mg, more preferably less than 50 mg, more preferably less than 20 mg, more preferably less than 10 mg of organic solvent per kg of β-lactam antibiotic.

5. Product according to claim 1, wherein said product is a crystalline powder of said β-lactam antibiotic.

6. Product according to claim 1, wherein said product is in the form of granules comprising said β-lactam antibiotic.

7. Product according to claim 1, wherein said product is free of auxiliaries.

8. Product according to claim 1, wherein said β-lactam antibiotic is a cephalosporin, preferably selected from the group consisting of cephalexin, cefadroxil, cephradin and cefaclor.

9. Product according to claim 1, wherein said β-lactam antibiotic is a penicillin, preferably selected from the group consisting of amoxicillin and ampicillin.

10. Product according to claim 1, wherein C₄₀₀≦0.20, wherein C₄₀₀ is the color measured at a wavelength of 400 nm (Ph. Eur.).

11. Product according to claim 1, wherein the water content (Karl Fisher) is less than 15%, for instance less than 8%.

12. Product according to claim 11, wherein the β-lactam antibiotic is cephalexin, and wherein the water content (Karl Fisher) is between 4.0 and 8.0%.

13. Product according to claim 11, wherein the β-lactam antibiotic is cefadroxil, and wherein the water content (Karl Fisher) is between 4.2 and 6.0%. 5.

14. Crystalline cephalexin monohydrate powder having a bulk density of above 0.25 g/ml, preferably above 0.30 g/ml, more preferably above 0.35 g/ml.

15. Process for drying a β-lactam antibiotic, for instance a cephalosporin, said process comprising drying said β-lactam antibiotic under decreased pressure.

16. Process according to claim 15, wherein the said drying is effected at a o pressure of between 1 and 500 mbar, preferably between 5 and 200 mbar, more preferably between 10 and 100 mbar.

17. Process according to claim 15, wherein said drying is effected at a temperature of between 40 and 80° C., preferably between 50 and 70° C.

18. Process, for instance according to claim 15, wherein said process comprises stirring said β-lactam antibiotic during said drying, preferably using mechanical means.

19. Process according to claim 18, wherein said drying is effected in a drying chamber, and wherein said stirring is effected using an agitator that is present in the drying chamber.

20. Process according to claim 19, wherein said agitator comprises an axis around which the agitator can be rotated in the drying chamber.

21. Process according to claim 18, wherein said agitator comprises blades connected to the axis.

22. Process according to claim 15, wherein said drying is effected in a contact dryer.

23. Process according to claim 15, wherein the process comprises drying crystals of said β-lactam antibiotic, said crystals having an aspect ratio higher than 3, for instance cephalexin crystals.

24. Process according to claim 15, wherein said β-lactam antibiotic is a cephalosporin, preferably selected from the group consisting of cephalexin, cefadroxil, cephradin and cefaclor.

25. Process according to claim 15, wherein said β-lactam antibiotic is a penicillin, preferably selected from the group consisting of amoxicillin and ampicillin.

26. Process according to claim 15, wherein said β-lactam antibiotic contains an organic solvent.

27. Process according to claim 26, wherein said organic solvent is a ketone, for instance acetone, an alcohol, or dichloromethane.

28. Process according to claim 15, wherein said process comprises washing the β-lactam antibiotic prior to said drying.

29. Process according to claim 15, wherein said process comprises washing the β-lactam antibiotic with acetone or an alcohol or with a solution containing acetone or an alcohol.

30. Process according to claim 15, wherein said process comprises washing the β-lactam antibiotic with water.

31. β-lactam antibiotic obtainable by the process according to claim 15.
